(19) United States
(12) Patent Application Publication
(10) Pub. No.: US 2003/0064396 A1
(43) Pub. Date: Apr. 3, 2003

(54) Co-modulators of nuclear receptors

(76) Inventors: Maik Obendorf, Weimar (DE); Siegmund Wolf, Jena (DE)

Correspondence Address: STRIKER, STRIKER & STENBY, 103 East Neck Road, Huntington, NY, 11743, US

(21) Appl. No.: 10/193,874

(22) Filed: Jul. 12, 2002

Related U.S. Application Data
(60) Provisional application No. 60/311,699, filed on Aug. 10, 2001.

(30) Foreign Application Priority Data
Jul. 23, 2001 (DE) 101 35 787.7

(51) Int. Cl. C12Q 1/68; C07H 21/04; C12N 9/00; C12P 21/02; C12N 5/06

(52) U.S. Cl. 435/6; 435/69.1; 435/183; 435/320.1; 435/325; 530/350; 536/23.2

(57) ABSTRACT

A testing system for identifying effectors of polypeptides (ARAP3 variants) having the respective amino acid sequences represented in Seq. ID Nos. 14 to 18 and 20. In this test system a reporter gene is expressed in a cell transfected with a nucleic acid or in a cell transfected with a vector containing at least one copy of this nucleic acid; the cell, if it contains no nuclear receptor or only a small amount thereof, is also transfected with a vector containing DNA of the nuclear receptor; the cell is cultured in the presence of a plurality of test substances and a change in expression of the reporter gene is measured to find the effectors. The nucleic acid codes one of the polypeptides (ARAP3 variants), or has a nucleotide sequence represented in one of Seq. ID Nos. 1 to 13 and 19, or has a nucleotide sequence hybridized with one of Seq. ID Nos. 1 to 13 and 19 and/or one of the nucleic acid sequences coding polypeptides with an amino acid sequence represented in one of Seq. ID Nos. 14 to 18 and 20 under stringent conditions or has a degenerated nucleotide sequence corresponding to one of the foregoing nucleotide sequences within the framework of the degeneration of the genetic code.

### CROSS-REFERENCE

This disclosure contains subject matter in common with U.S. provisional application, Ser. No. 60/311,699, filed Aug. 10, 2001.

### REFERENCE TO SEQUENCE LISTING TABLES

Sequence listing tables are appended hereinbelow. First sequence listing tables list sequences for nucleic acids of the invention designated as Seq. ID Nos. 1 to 13 and 19. Second or following sequence listing tables list the amino acid sequences of polypeptides of the invention designated Seq. ID Nos. 14 to 18 and 20.

### BACKGROUND OF THE INVENTION

1. Field of the Invention

The invention relates to co-modulators of nuclear receptors and the use thereof for preparing novel drugs.

2. Description of the Related Art

The super-family of nuclear receptors, which includes about 50 different proteins, consists of a group of related transcription factors, which control the transcription of a particular target gene as a function of certain specific ligands. On the basis of certain criteria, for example dimerization status, type of ligand or structure of the DNA binding element this family can be subdivided into several subfamilies (Beato et al., 2000, Human Reproduct. Update 6, 225-236). A characteristic feature of nuclear receptors is the matching structure of functional domains (marked A to F) consisting of a highly variable, only slightly preserved N-terminal region with autonomous constitutive activation function (AF-1), a well preserved DNA-binding domain (DBD), which is responsible for recognition of special DNA-binding elements and consists of two zinc finger motifs, a variable hinge domain and a preserved multifunctional C-terminal ligand-binding domain (LBD) consisting of a dimerization-dependent and ligand-dependent transactivation function (AF-2). This is followed by the region located at the most remote C-terminal whose function is not known and which is absent in receptors such as, for example, PR (progesterone receptor), PPAR (peroxisome proliferator-activated receptor) and RXR (retinoid X receptor) (Mangelsdorf & Evans, 1995, Cell 83, 841-850; Robyr et al., 2000, Mol. Endocrinol. 14, 329-347). It was demonstrated for some nuclear receptors (for example AR) that the N-terminal region is able to interact with the C-terminal region (Brinkmann et al., 1999, J. Steroid Biochem. and Mol. Biol. 69, 307-313). Steroid hormone receptors such as, for example, estrogen receptors (ER), progesterone receptors (PR), glucocorticoid receptors (GR), mineralocorticoid receptors (MR) and androgen receptors (AR) bind steroid ligands, such as the progestins, estrogens, glucocorticoids, mineralocorticoids and androgens all of which are derived from pregnenolone. The binding of the ligands to NR activates the receptor and controls the expression of the corresponding target genes.

Moreover, another class of proteins, known as co-modulators, has been identified. These proteins play an important role either in the activation (co-activators) or in the repression (co-repressors) of gene transcription as bridging molecules between the transcription initiation complex and the nuclear receptors (McKenna et al., 1999, Endocr. Rev. 20, 321-347). A co-activator enhances the receptor function and in the presence of an agonist—but not in the presence of an antagonist—interacts directly with the activation domains of nuclear receptors. It also interacts with the basal transcription apparatus, but does not spontaneously enhance the basal transcription activity. Most co-modulators interact with the AF-2 domain of nuclear receptors with the aid of one or more LXXLL motifs (NR boxes) in the protein sequence. Several co-modulators, however, which can interact with other amino acids with nuclear receptors (Ding et al. 1998, Mol. Endocrinol. 12, 302-313) have been described. Moreover, many co-modulators, which in similar manner interact with several different nuclear receptors have been identified.

It was shown for an estrogen receptor co-activator, known as A1B1, that it is expressed to a higher degree in cell lines of breast cancer and ovarian cancer and presumably plays an important role in the development of steroid hormone-dependent tumors (Anzick et al. 1997, Science 277, 965-968).

### SUMMARY OF THE INVENTION

Besides influencing steroid hormone receptors directly with hormones or antihormones, modulating the interaction of co-activators with the steroid hormone receptors could be another approach to therapy for hormone-dependent diseases. The problem, therefore, is to find a new co-activator whereby novel drugs can be provided.

This problem was solved by preparing nucleic acids consisting of

a) nucleic acids that code for polypeptides that have the amino acid sequences represented in Sequence ID Nos. 14 to 18 and 20,

b) nucleic acids that have the nucleotide sequences represented in Sequence ID Nos. 1 to 13 and 19,

c) nucleic acids with nucleotide sequences hybridizing with one of the sequences from a) and/or b) under stringent conditions and which code for polypeptides with the biological activity of a co-activator, or

d) nucleic acids with nucleotide sequences corresponding to the sequences from a), b) or c) within the framework of degeneration of the genetic code.

Fragments of these sequences have been published in the GenBank databank under Accession Nos. AB037801, AK027280 and AK024991. No functions were assigned to these fragments.

The concept “hybridization under stringent conditions” according to the present invention is defined by Sambrook et al. (Molecular Cloning. A Laboratory Manual, Cold Spring Harbor Laboratory Press, 1989). Stringent hybridization is present, for example, when after 1-hour washing with 1×SSC and 0.1% SDS at 50° C., preferably at 55° C., particularly at 62° C. and most preferably at 68° C., preferably for one hour in 0.2×SSC and 0.1% SDS at 55° C., particularly at 62° C. and most preferably at 68° C., a hybridization signal is still observed (SSC means standard saline citrate solution; SDS means sodium dodecylsulfate). The nucleic acids, which under these conditions hybridize with the nucleic acids represented in Sequence ID Nos. 1 to 13 and 19, or with one of the degenerate sequences within the framework of degeneration of the genetic code, are also an object of the present invention.

The nucleic acids can be a single-stranded or double-stranded DNA, for example cDNA, or an RNA, for example mRNA, cRNA or pre-mRNA.

The invention also concerns polypeptides coded by nucleic acids of the invention or having the amino acid sequences represented in Sequence ID Nos. 14 to 18 and 20. In the following, these polypeptides of the invention are referred to as ARAP3 variants or, briefly, as ARAP3. These nucleic acid variants are expressed by the use of alternative promoters and by alternative processing or splicing of the pre-mRNA. ARAP3 variants contain a zinc finger domain. They show homologies to other members of the zinc finger domain family such as, for example, hairless protein (Cachon-Gonzales et al., 1994, Proc. Natl. Acad. Sci. 91, 7717-7721) and testis-specific protein a (Hoog et al., Mol. Reprod. Dev. 30,173-181). Moreover, the C-terminal amino acids have significant homologies to the jmjC domain of the jumonji family (Balciunas and Ronne, 2000, Trends Biochem. Sci. 25, 274-76). ARAP3 also contains a nucleus localization domain. ARAP3 binds to the androgen receptor. The binding site is located in the region of amino acids 325 to 919 of the androgen receptor (see FIG. 2, AR2 fragment). The binding sites for the polypeptides of the invention are located as shown in the following table:

TABLE I
AMINO ACID REGIONS OF THE DOMAINS OF ARAP3
POLYPEPTIDES

| | | Androgen | | |
|---|---|---|---|---|
| | | receptor | Nucleus | |
| Seq. | Zinc | Binding | Localization | |
| ID No. | Finger AS | Domain | Domain (NLD) | JmjC-Domain |
| 14 | 1664-1692 | 1732-1841 | 2183-2193 | 2199-2298 |
| 15 | 1858-1886 | 1926-2035 | 2377-2385 | 2393-2492 |
| 16 | 1846-1874 | 1914-2023 | 2365-2375 | 2381-2480 |
| 17 | 1627-1655 | 1695-1804 | 2146-2154 | 2162-2261 |
| 18 | 390-418 | 458-567 | 909-917 | 925-953 part. |
| 20 | 1361-1389 | 1430-1539 | 1881-1898 | 1897-1996 |

ARAP3 has the function of a co-modulator of nuclear receptors, particularly steroid hormone receptors. Examples of such receptors are the androgen receptor, estrogen receptor α, estrogen receptor β, progesterone receptor A, progesterone receptor B, glucocorticoid receptor, mineralocorticoid receptor, thyroid hormone receptor, vitamin D receptor and peroxisome proliferator-activated receptor. ARAP3 binds particularly well to the androgen receptor thereby enhancing the receptor function. The receptor function can be modulated, i.e. strengthened or weakened, by the binding.

In healthy humans, ARAP3 is expressed especially strongly in the heart, liver, testicles and ovaries.

Another object of the invention are vectors containing at least one copy of a nucleic acid of the invention. The vectors can be prokaryotic or eukaryotic vectors. Examples of vectors are pPRO (Clontech), pBAD (Invitrogen), pSG5 (Stratagene), PCI (Promega), pIRES (Clontech), pBAC (Clontech), pMET (Invitrogen) and pBlueBac (Invitrogen). By methods known to those skilled in the art, the nucleic acids of the invention can be inserted into these vectors. The nucleic acids of the invention are preferentially linked to the expression signals such as, for example, promotor and enhancer on the vector.

The invention also relates to cells transfected with nucleic acid sequences of the invention or with a vector of the invention. For, example, E. coli, yeast, Pichia, Sf9, COS, CV-1 or BHK can be used as the cells. These cells can be employed for the production of the polypeptides of the invention or for testing systems.

The polypeptides of the invention or partial regions thereof (peptides) can be used for the preparation of antibodies. To produce polyclonal antibodies, the polypeptides or peptides, for example KLH (keyhole limpet hemocyanin) can be linked and injected into animals, for example rabbits. They can also be used for preparing monoclonal antibodies. To prepare the antibodies, a polypeptide or peptide of the invention or a mixture of several peptides of the invention can be used. The antibodies are prepared by standard procedures, as described, for example, by Kohler, G. and Milstein, C., Nature 1975, 265, 495-497, and by Nelson, P.N. et al., Mol. Pathol. 2000; 53, 111-117.

Yet another object of the invention are antibodies directed against the polypeptides of the invention.

The antibodies of the invention can be used for detecting ARAP3 and its variants. This can be done, for example, by immunohistochemistry. The antibodies of the invention can also be used in other immune tests, for example ELISA (enzyme linked immunosorbent assay) or in radjoimmune tests. In this manner, the concentration of ARAP3 can be determined in tissue extracts or cell extracts.

Detection of expression of the polypeptides of the invention can also be accomplished through the detection of mRNA in the cells. Hence, an object of the invention is also the use of a probe with nucleic acid sequences that are complementary to the nucleic acid sequences coding for ARAP3, for preparing a reagent for detecting the presence of the mRNA of the invention in cells. A probe is a short piece of DNA with at least 14 nucleotides. The probes of the invention can be used, for example, in a Northern blot analysis. This method is described, for example, in Sambrook, J., et al., 1989, Cold Spring Harbor Laboratory Press. Other methods for detecting RNA are in-site hybridization, RNAse protection assay or polymer chain reaction (PCR).

The detection or identification of ARAP3 expression is used primarily in androgen-dependent diseases. For example, elevated androgen activation in diseases, such as prostate cancer and benign prostate tumors and in acne or loss of hair, can be attributed to increased co-activator activity of ARAP3. On the other hand, a reduced co-activator activity can be present in hypogonadism, erectile dysfunction and androgen insensitive syndromes, for example the testicular feminization syndrome. In these diseases, the balance of androgen receptor and co-activator can be disturbed. Hence, it is advantageous to determine, besides the amount of expressed ARAP3, also the amount of expressed androgen receptor in the same tissue. The androgen receptor protein can also be determined by immunological methods, for example by radioimmunoassay, ELISA or Western blot.

Another object of the invention is the use of ARAP3 or of the nucleic acids that code for it or its variants as target substance for preparing an agent for treating steroid hormone-dependent diseases. Such steroid hormone-dependent diseases also include besides the above-said androgen-dependent diseases, for example, estrogen-dependent diseases such as breast cancer, and osteoporosis or cardiovascular diseases and vascular diseases. ARAP3 could also be used as target substance for the preparation of drugs to influence male fertility.

In particular, the invention comprises the use of

a) a nucleic acid of the invention

b) a polypeptide of the invention or

c) a cell of the invention

for the purpose of identifying effectors of ARAP3.

Effectors are substances with an inhibitory or activating effect on ARAP3 and which are capable of influencing the co-activator function of ARAP3. Preferred are substances, which modulate the interaction of ARAP3 with the androgen receptor. This can be tested, for example, by measuring the binding of the purified ARAP3 polypeptide to the androgen receptor polypeptide in the presence of the test substance and comparing the result with the control value obtained without the test substance. The binding can be determined with the aid of a marker bound to ARAP3 or to the androgen receptor. Such markers can be, for example, fluorescence markers, biotin or radioactive markers.

Another possibility of identifying effectors consists of using a cell containing the cDNA of ARAP3 or of a functional part thereof, the cDNA of the androgen receptor or some other nuclear receptor and the cDNA of a reporter gene.

The reporter gene used can be, for example, luciferase. The activity of luciferase in this case reflects the activity of the nuclear receptor. The cells are incubated in the presence of the test substance, and the luciferase activity is determined. The incubation can also be carried out in the additional presence of the ligand of the nuclear receptor. For example, antagonistic effects can be measured in this manner. The use of the androgen receptor and its ligand, an androgen, is preferred.

The effectors of ARAP3 can be used for the treatment of steroid hormone-dependent diseases. In diseases involving, for example, strong androgen activation, an inhibitor of the interaction of ARAP3 and the androgen receptor can be administered, and in diseases that involve reduced co-activator activity, a stimulator of this interaction can be administered.

Diseases involving a deficiency of ARAP3 can be treated also by modifying the ARAP3 concentration in the affected tissues. To this end, into the tissue is introduced either a nucleic acid of the invention with the aid of a vector used in gene therapy, or a polypeptide of the invention. In gene therapy, a vector containing a nucleic acid of the invention is constructed and administered. Examples are vectors derived from adenovirus, adenovirus-associated virus, herpes simplex virus or SV40. The gene therapy can be carried out by a protocol such as that described by Gomez-Navarro, J. et al. (Eur. J. Cancer 1999, 35, 867-885). Administration can be local, namely directly into the affected tissue, for example the tumor, or systemically, namely via the blood circulation. This results in increased expression of ARAP3.

ARAP3 can be administered in the form of a fusion polypeptide. With the aid of the fused polypeptide, for example EGF [epidermal growth factor] or transferrin, the polypeptide of the invention is transported preferentially into the desired tissue, for example the tumor tissue.

Diseases can also be due to excessive expression of ARAP3. In this case, the nuclear receptors are so highly sensitized that they can be activated not only by their ligands but also by other substances, which show no effect under physiological conditions. In this case, it is desirable to reduce the expression of ARAP3. This can be done with antisense molecules. These molecules are complementary to the nucleic acid sequences represented in Seq. ID Nos. 1 to 13 and 19, or parts thereof.

Yet another object of the invention is a method for preparing a pharmaceutical agent, wherein

a) substances are brought in contact with a testing system of the invention,

b) the action of the substances on the testing system is measured by comparison with a control,

c) a substance which in step b) showed modulation of ARAP3 activity is identified, and

d) the substance identified in step c) is mixed with formulation materials commonly used for pharmaceuticals.

The activity of ARAP3, measured in step b), is represented by the enhancement of the receptor function of the nuclear receptor used. Preferred are steroid hormone receptors, the androgen receptor being particularly preferred.

The testing systems of the invention can also be used to test environmental samples. Many substances in the environment occur in such low concentrations that they exert an effect only on human steroid hormone receptors, which express the corresponding co-activator in increased amounts. By use of a testing system of the invention, these substances can be identified. A genetic predisposition to the action of these substances can be determined by ARAP3 detection according to the invention.

### BRIEF DESCRIPTION OF THE SEVERAL VIEWS OF THE DRAWING

The objects, features and advantages of the invention will now be illustrated in more detail with the aid of the following examples, with reference to the accompanying figures in which:

FIG. 1 is a schematic representation of the androgen receptor, in which AF stands for activation function, DBD for the DNA binding domain, LBD for the ligand-binding domain and AS for amino acids and which shows the fragment of the androgen receptor (AR2), which was used for the two-hybrid screen;

FIG. 2 is a schematic representation of the ARAP3 polypeptide Seq. ID No. 20, which shows the region binding to the androgen receptor and the zinc finger domain;

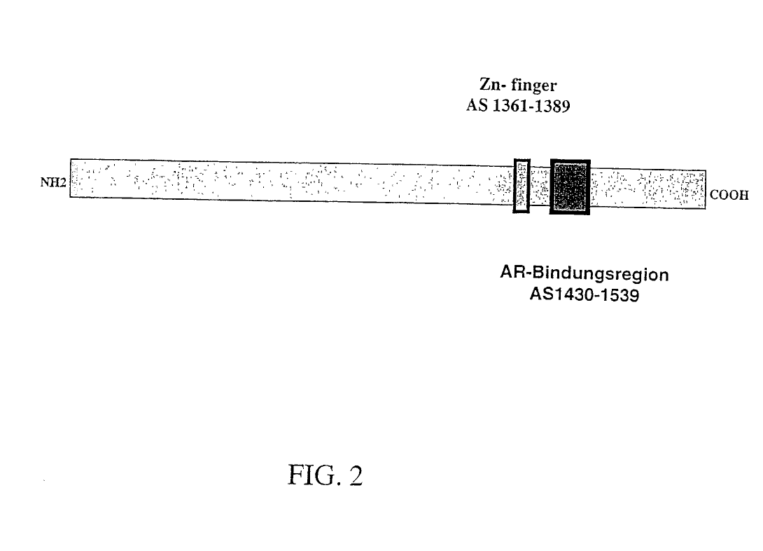

FIG. 3 shows the tissue distribution of ARAP3 in a Northern blot analysis in which 2 μg of human poly A+RNA was separated on a gel, transferred to a membrane and hybridized with a ARAP3-cDNA fragment (kb stands for kilobases); and

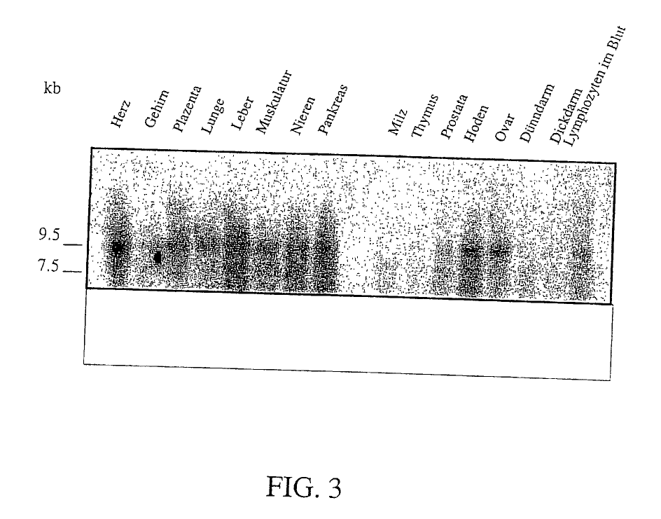

FIG. 4 shows the result of a mammal hybrid test with the ARAP3 fragment AS 1430 to 1539 of Seq. ID No. 20 and the human androgen receptor, which was performed as described in Example 2 below and which employed an analogous VP16 construct of an SRC1 fragment binding to the androgen receptor as positive control.

### EXAMPLES

### Example 1

### Two-Hybrid Screen

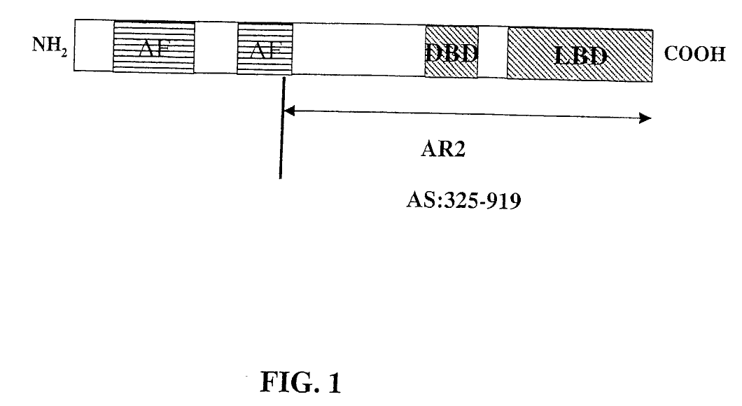

By use of a cDNA library from fetal brain (Clontech MATCHMAKER) and a human AR fragment coding for the amino acids 325 to 919 as the probe (FIG. 1), a screening was performed by means of the yeast-2-hybrid system in the presence and in the absence of 10⁻⁶ mol of dihydroxytestosterone (DHT). In accordance with the producer's instructions (Clontech), the number of screened clones was 3×10 ⁶ and 2×10⁷. According to the producer's information, the number of independent clones was 3.5×10⁶. From these, we selected 800 positive clones and tested them by a β-galactosidase assay, which confirmed 240 as lacZ-positive clones. The inserts of these clones were amplified by PCR. By means of restriction fragment analysis and sequencing, at least 17 different clones were identified. One of these was a clone with an insert comprising 327 base pairs coding for part of the ORF (open reading frame) of KIAA1380 (gene bank access number AB03780 1). In screening the library with and without 10⁻⁶ mol of DHT, this clone was identified forty times.

With the aid of PCR techniques, ARAP3-cDNA was then lengthened. Different splicing variants were found for them, which represent the nucleic acids having the nucleic acid sequences Seq. ID Nos. 1 to 13. From these five polypeptides were derived with the amino acid sequences Seq. ID Nos. 14 to 18.

### Example 2

### Mammal Hybrid Test

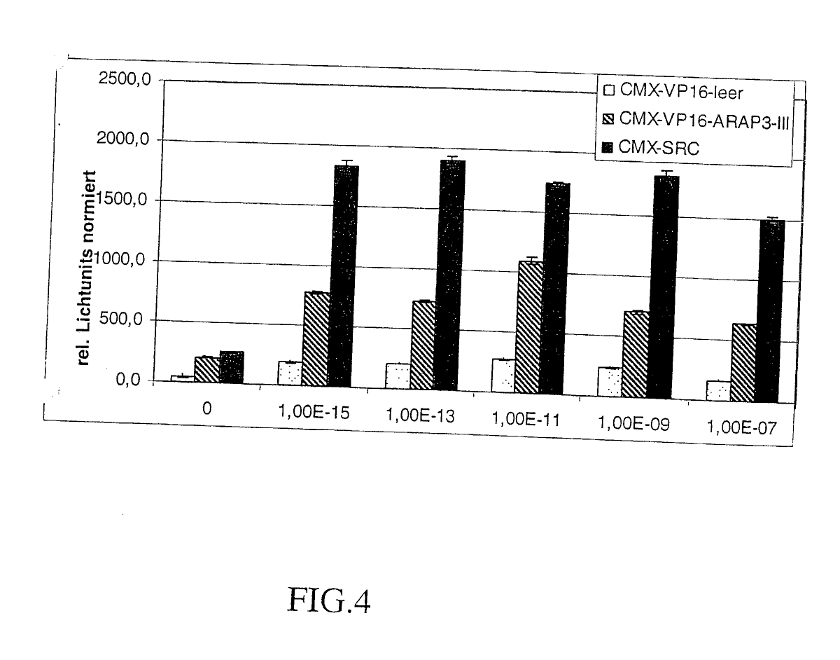

The binding of the ARAP3 fragment (AS 1430-1539) to the androgen receptor (AR) was confirmed by the mammal hybrid test (FIG. 4). The ARAP3 fragment as fusion protein was cloned with VP16 (CMX VP1 6-ARAP3-III). Human PC3 cells were transfected with CMX-VP1 6-ARAP3-III, the expression vector that contains the complete AR, (pSG5AR) and with the reporter gene, luciferase, which is under the control of the AR-dependent MMTV promoter (pMMTV-luc). After 24 hours, the cells were incubated with dihydroxytestosterone (DHT). After an additional day, the cells were lysed, and the activity of the reported gene luciferase was determined. The results were normalized by means of the protein content of the preparations, determined in parallel. The control used was a preparation with the empty expression vector CMX-VP1 6-empty.

### Example 3

### Determination of the Co-Activator Activity of ARAP3

An eukaryotic expression vector, for example pCMX which contains ARAP3-cDNA that codes for the complete ARAP3 protein or for a functional part thereof (PCMX-ARAP3), was transfected in suitable cell lines, for example SH-SY5Y or PC3, together with pSG5AR and pMMTV-luc. The control was a preparation with the empty expression vector pCMX. The androgen activity can be determined from the activity of the reporter luciferase as in the foregoing example. The effect of ARAP3 on the androgenic signal can be determined by comparison with the activity of the control preparation.

### Example 4

### Determination of the Binding of ARAP3 to the Androgen Receptor by Means of a Pull-Down Experiment

A GST pull-down experiment is characterized by an experimental procedure, which allows binding of an in vitro expressed GST fusion protein to a similar in vitro expressed and radioactively labeled protein, subsequent separation from non-interacting proteins and detection and identification of the bound protein as a measure of the binding. The experiments were performed according to the protocol as described in Sambrook and Russel, Molecular Cloning, Volume 3, Chapter 18, Procedure 3 (p. 18.55 ff); Cold Spring Harbor Laboratory Press, New York, 2001.

For the expression an ARAP3 fragment (AS 1735 to 1840 of Seq. ID No. 14) was cloned as a GST fusion protein in the vector pGEX-KG (Pharmacia) and was over-expressed and prepared in bacterial cells according to the procedure of the manufacturer. The ARAP3 fusion protein was bound to glutathione sepharose spherical particles and incubated with a 35S-methionine-labeled androgen receptor (pSG5AR with T7 TNT reticulozyte lysate of Promega). Subsequently this was washed with centrifuging. The bound portion of the androgen receptor to the GST-ARAP3 spherical particles was determined by SDS-PAGE and subsequent autofluorography. The binding ratio could be formed in a similar treatment of non-fusioned GST spherical particles relative to the GST-ARAP3 spherical particles in an experimental start by comparison of the blackening of the androgen receptor protein band on the hyperfilm (TM Amersham) and densitometry analysis by means of density integration. Validated binding ratio values for GST-ARAP3 to GST-empty resulted. These binding ratios are tabulated in Table II below and shown with the amounts of protein used as determined by Western Blot. These binding ratios are regarded as detection or proof of the binding of ARAP3 to the androgen receptor.

TABLE II

| | Protein, Western | | Binding, Pull-down | | Ratio |
|---|---|---|---|---|---|
| | GST- | GST- | | GST- | GST-ARAP3/ |
| Exp. | empty In | ARAP3 | GST-empty | ARAP3 | GST-empty |
| No. | Vol./μl | In Vol./μl | In Vol./μl | In Vol./μl | Ratio |
| 1 | 445 | 220 | 160 | 2094 | 26.5 |
| 2 | 833 | 351 | 10.6 | ≈5000 | 1122 |
| 3 | 4614 | 208 | <1 | 257 | >>100 |
| 4 | 349 | 382 | <1 | 225 | >>100 |

The disclosure in German Patent Application 101 35 787.7 of Jul. 23, 2001 is incorporated here by reference. This German Patent Application describes the invention described hereinabove and claimed in the claims appended hereinbelow and provides the basis for a claim of priority for the instant invention under 35 U.S.C. 119.

While the invention has been illustrated and described as embodied in compositions for transferring active compounds in a cell-specific manner, it is not intended to be limited to the details shown, since various modifications and changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed is new and is set forth in the following appended claims.

We claim:

1. A nucleic acid consisting of
a) a nucleic acid that codes a polypeptide that includes one of amino acid sequences represented in Seq. ID Nos. 14 to 18 and 20,
b) a nucleic acid that includes one of nucleotide sequences represented in Seq. ID Nos. 1 to 13 and 19,
c) a nucleic acid having a hybridized nucleotide sequence hybridized with at least one of said nucleotide sequences represented in said Seq. ID Nos. 1 to 13 and 19 and said nucleotide sequences of said nucleic acid coding said polypeptide that includes one of said amino acid sequences represented in said Seq. ID Nos. 14 to 18 and 20 under stringent conditions and wherein said hybridized nucleotide sequence codes for a polypeptide with biological activity of a co-modulator, or
d) a nucleic acid having a degenerate nucleotide sequence corresponding to one of said nucleotide sequences represented in said Seq. ID Nos. 1 to 13 and 19, said nucleotide sequence of said nucleic acid coding said polypeptide that includes one of said amino acid sequences represented in said Seq. ID Nos. 14 to 18 and 20 and said hybridized nucleotide sequence within the framework of degeneration of the genetic code.

2. A polypeptide coded by a nucleic acid, said nucleic acid consisting of
a) a nucleic acid that codes a polypeptide that includes one of amino acid sequences represented in Seq. ID Nos. 14 to 18 and 20,
b) a nucleic acid that includes one of nucleotide sequences represented in Seq. ID Nos. 1 to 13 and 19,
c) a nucleic acid having a hybridized nucleotide sequence hybridized with at least one of said nucleotide sequences represented in said Seq. ID Nos. 1 to 13 and 19 and said nucleotide sequences of said nucleic acid coding said polypeptide that includes one of said amino acid sequences represented in said Seq. ID Nos. 14 to 18 and 20 under stringent conditions and wherein said hybridized nucleotide sequence codes for a polypeptide with biological activity of a co-modulator, or
d) a nucleic acid having a degenerate nucleotide sequence corresponding to one of said nucleotide sequences represented in said Seq. ID Nos. 1 to 13 and 19, said nucleotide sequence of said nucleic acid coding said polypeptide that includes one of said amino acid sequences represented in said Seq. ID Nos. 14 to 18 and 20 and said hybridized nucleotide sequence within the framework of degeneration of the genetic code.

3. A polypeptide comprising an amino acid sequence represented in one of Seq. ID Nos. 14 to 18 and 20.

4. A vector containing at least one copy of a nucleic acid, said nucleic acid consisting of
a) a nucleic acid that codes a polypeptide that includes one of amino acid sequences represented in Seq. ID Nos. 14 to 18 and 20,
b) a nucleic acid that includes one of nucleotide sequences represented in Seq. ID Nos. 1 to 13 and 19,
c) a nucleic acid having a hybridized nucleotide sequence hybridized with at least one of said nucleotide sequences represented in said Seq. ID Nos. 1 to 13 and 19 and said nucleotide sequences of said nucleic acid coding said polypeptide that includes one of said amino acid sequences represented in said Seq. ID Nos. 14 to 18 and 20 under stringent conditions and wherein said hybridized nucleotide sequence codes for a polypeptide with biological activity of a co-modulator, or
d) a nucleic acid having a degenerate nucleotide sequence corresponding to one of said nucleotide sequences represented in said Seq. ID Nos. 1 to 13 and 19, said nucleotide sequence of said nucleic acid coding said polypeptide that includes one of said amino acid sequences represented in said Seq. ID Nos. 14 to 18 and 20 and said hybridized nucleotide sequence within the framework of degeneration of the genetic code.

5. A cell transfected with a nucleic acid or transfected with a vector containing at least one copy of said nucleic acid, wherein said nucleic acid consists of
a) a nucleic acid that codes a polypeptide that includes one of amino acid sequences represented in Seq. ID Nos. 14 to 18 and 20,
b) a nucleic acid that includes one of nucleotide sequences represented in Seq. ID Nos. 1 to 13 and 19,
c) a nucleic acid having a hybridized nucleotide sequence hybridized with at least one of said nucleotide sequences represented in said Seq. ID Nos. 1 to 13 and 19 and said nucleotide sequences of said nucleic acid coding said polypeptide that includes one of said amino acid sequences represented in said Seq. ID Nos. 14 to 18 and 20 under stringent conditions and wherein said hybridized nucleotide sequence codes for a polypeptide with biological activity of a co-modulator, or
d) a nucleic acid having a degenerate nucleotide sequence corresponding to one of said nucleotide sequences represented in said Seq. ID Nos. 1 to 13 and 19, said nucleotide sequence of said nucleic acid coding said polypeptide that includes one of said amino acid sequences represented in said Seq. ID Nos. 14 to 18 and 20 and said hybridized nucleotide sequence within the framework of degeneration of the genetic code.

6. A method of expressing a nucleic acid consisting of
a) a nucleic acid that codes a polypeptide that includes one of amino acid sequences represented in Seq. ID Nos. 14 to 18 and 20,
b) a nucleic acid that includes one of nucleotide sequences represented in Seq. ID Nos. 1 to 13 and 19,
c) a nucleic acid having a hybridized nucleotide sequence hybridized with at least one of said nucleotide sequences represented in said Seq. ID Nos. 1 to 13 and 19 and said nucleotide sequences of said nucleic acid coding said polypeptide that includes one of said amino acid sequences represented in said Seq. ID Nos. 14 to 18 and 20 under stringent conditions and wherein said hybridized nucleotide sequence codes for a polypeptide with biological activity of a co-modulator, or
d) a nucleic acid having a degenerate nucleotide sequence corresponding to one of said nucleotide sequences represented in said Seq. ID Nos. 1 to 13 and 19, said nucleotide sequence of said nucleic acid coding said polypeptide that includes one of said amino acid sequences represented in said Seq. ID Nos. 14 to 18 and 20 and said hybridized nucleotide sequence within the framework of degeneration of the genetic code;
wherein said method comprises using a cell for the expressing.

7. A method of producing an antibody comprising producing a polypeptide or a part of said polypeptide, wherein said polypeptide or said part is coded by a nucleic acid, said nucleic acid consisting of
a) a nucleic acid that codes a polypeptide that includes one of amino acid sequences represented in Seq. ID Nos. 14 to 18 and 20,
b) a nucleic acid that includes one of nucleotide sequences represented in Seq. ID Nos. 1 to 13 and 19,
c) a nucleic acid having a hybridized nucleotide sequence hybridized with at least one of said nucleotide sequences represented in said Seq. ID Nos. 1 to 13 and 19 and said nucleotide sequences of said nucleic acid coding said polypeptide that includes one of said amino acid sequences represented in said Seq. ID Nos. 14 to 18 and 20 under stringent conditions and wherein said hybridized nucleotide sequence codes for a polypeptide with biological activity of a co-modulator, or
d) a nucleic acid having a degenerate nucleotide sequence corresponding to one of said nucleotide sequences represented in said Seq. ID Nos. 1 to 13 and 19, said nucleotide sequence of said nucleic acid coding said polypeptide that includes one of said amino acid sequences represented in said Seq. ID Nos. 14 to 18 and 20 and said hybridized nucleotide sequence within the framework of degeneration of the genetic code.

8. A method of producing an antibody comprising producing a polypeptide or a part of said polypeptide, wherein said polypeptide or said part of said polypeptide has an amino acid sequence represented in one of Seq. ID Nos. 14 to 18 and 20.

9. An antibody against a polypeptide or a part of said polypeptide, wherein said antibody is made by a method comprising producing a polypeptide or a part of said polypeptide, wherein said polypeptide or said part is coded by a nucleic acid, said nucleic acid consisting of
a) a nucleic acid that codes a polypeptide that includes one of amino acid sequences represented in Seq. ID Nos. 14 to 18 and 20,
b) a nucleic acid that includes one of nucleotide sequences represented in Seq. ID Nos. 1 to 13 and 19,
c) a nucleic acid having a hybridized nucleotide sequence hybridized with at least one of said nucleotide sequences represented in said Seq. ID Nos. 1 to 13 and 19 and said nucleotide sequences of said nucleic acid coding said polypeptide that includes one of said amino acid sequences represented in said Seq. ID Nos. 14 to 18 and 20 under stringent conditions and wherein said hybridized nucleotide sequence codes for a polypeptide with biological activity of a co-modulator, or
d) a nucleic acid having a degenerated nucleotide sequence corresponding to one of said nucleotide sequences represented in said Seq. ID Nos. 1 to 13 and 19, said nucleotide sequence of said nucleic acid coding said polypeptide that includes one of said amino acid sequences represented in said Seq. ID No. 14 to 18 and 20 and said hybridized nucleotide sequence within the framework of degeneration of the genetic code.

10. An antibody against a polypeptide or a part of said polypeptide, wherein said antibody is made by a method comprising producing a polypeptide or a part of said polypeptide, wherein said polypeptide or said part has an amino acid sequence represented in one of Seq. ID Nos. 14 to 18 and 20.

11. A method of detecting a polypeptide or a part of a polypeptide comprising detecting said polypeptide or said part of said polypeptide with an antibody against said polypeptide or said part of said polypeptide, wherein said polypeptide or said part is coded by a nucleic acid, said nucleic acid consisting of
a) a nucleic acid that codes a polypeptide that includes one of amino acid sequences represented in Seq. ID Nos. 14 to 18 and 20,
b) a nucleic acid that includes one of nucleotide sequences represented in Seq. ID Nos. 1 to 13 and 19,
c) a nucleic acid having a hybridized nucleotide sequence hybridized with at least one of said nucleotide sequences represented in said Seq. ID Nos. 1 to 13 and 19 and said nucleotide sequences of said nucleic acid coding said polypeptide that includes one of said amino acid sequences represented in said Seq. ID Nos. 14 to 18 and 20 under stringent conditions and wherein said hybridized nucleotide sequence codes for a polypeptide with biological activity of a co-modulator, or
d) a nucleic acid having a degenerate nucleotide sequence corresponding to one of said nucleotide sequences represented in said Seq. ID Nos. 1 to 13 and 19, said nucleotide sequence of said nucleic acid coding said polypeptide that includes one of said amino acid sequences represented in said Seq. ID No. 14 to 18 and 20 and said hybridized nucleotide sequence within the framework of degeneration of the genetic code.

12. A method of detecting a polypeptide or a part of a polypeptide comprising detecting said polypeptide or said part of said polypeptide with an antibody against said polypeptide or said part of said polypeptide, wherein said polypeptide or said part has an amino acid sequence represented in one of Seq. ID Nos. 14 to 18 and 20.

13. A method of preparation of a reagent for detection of mRNA comprising testing for the mRNA with a probe having a nucleic acid sequence complementary to a member selected from the group consisting of nucleotide sequences represented in Seq. ID Nos. 1 to 13 and 19, nucleotide sequences that code polypeptides that have amino acid sequences represented in Seq. ID Nos. 14 to 18 and 20, hybridized nucleotide sequences hybridized with said nucleotide sequences represented in said Seq. ID Nos. 1 to 13 and 19 and with said nucleotide sequences that code said polypeptides under stringent conditions and degenerate nucleotide sequences corresponding to one of the foregoing said nucleotide sequences within the framework of genetic code degeneration.

14. A method of preparation of a reagent for detection of mRNA comprising testing for the mRNA with a probe having nucleic acid sequences complementary to nucleotide sequences that code for polypeptides, or parts of polypeptides having amino acid sequences represented in Seq. ID Nos. 14 to 18 and 20.

15. A method of producing an effective agent for treating steroid hormone-dependent diseases, said method comprising using a nucleic acid as a target substance for producing said agent and wherein said nucleic acid consists of
a) a nucleic acid that codes a polypeptide that includes one of amino acid sequences represented in Seq. ID Nos. 14 to 18 and 20,
b) a nucleic acid that includes one of nucleotide sequences represented in Seq. ID Nos. 1 to 13 and 19,
c) a nucleic acid having a hybridized nucleotide sequence hybridized with at least one of said nucleotide sequences represented in said Seq. ID Nos. 1 to 13 and 19 and said nucleotide sequences of said nucleic acid coding said polypeptide that includes one of said amino acid sequences represented in said Seq. ID Nos. 14 to 18 and 20 under stringent conditions and wherein said hybridized nucleotide sequence codes for a polypeptide with biological activity of a co-modulator, or
d) a nucleic acid having a degenerate nucleotide sequence corresponding to one of said nucleotide sequences represented in said Seq. ID Nos. 1 to 13 and 19, said nucleotide sequence of said nucleic acid coding said polypeptide that includes one of said amino acid sequences represented in said Seq. ID Nos. 14 to 18 and 20 and said hybridized nucleotide sequence within the framework of degeneration of the genetic code.

16. A method of producing an effective agent for treating steroid hormone-dependent diseases, said method comprising using a polypeptide a target substance for producing said agent and wherein said polypeptide or a part of said polypeptide is coded by a nucleic acid, said nucleic acid consisting of
a) a nucleic acid that codes a polypeptide that includes one of amino acid sequences represented in Seq. ID Nos. 14 to 18 and 20,
b) a nucleic acid that includes one of nucleotide sequences represented in Seq. ID Nos. 1 to 13 and 19,
c) a nucleic acid having a hybridized nucleotide sequence hybridized with at least one of said nucleotide sequences represented in said Seq. ID Nos. 1 to 13 and 19 and said nucleotide sequences of said nucleic acid coding said polypeptide that includes one of said amino acid sequences represented in said Seq. ID Nos. 14 to 18 and 20 under stringent conditions and wherein said hybridized nucleotide sequence codes for a polypeptide with biological activity of a co-modulator, or
d) a nucleic acid having a degenerate nucleotide sequence corresponding to one of said nucleotide sequences represented in said Seq. ID Nos. 1 to 13 and 19, said nucleotide sequence of said nucleic acid coding said polypeptide that includes one of said amino acid sequences represented in said Seq. ID Nos. 14 to 18 and 20 and said hybridized nucleotide sequence within the framework of degeneration of the genetic code.

17. A method of identifying an effector of a polypeptide, said polypeptide including an amino acid sequence, which is represented in one of Seq. ID Nos. 14 to 18 and 20 or which is coded by a nucleic acid that codes said polypeptide, a nucleic acid that includes one of nucleotide sequences represented in Seq. ID Nos. 1 to 13 and 19, a nucleic acid having a hybridized nucleotide sequence hybridized with at least one of said nucleotide sequences represented in said Seq. ID Nos. 1 to 13 and 19 and with said nucleotide sequences of said nucleic acid coding said polypeptide under stringent conditions, said hybridized nucleotide sequence coding for a polypeptide with biological activity of a co-modulator, or a nucleic acid having a degenerate nucleotide sequence corresponding to one of said nucleotide sequences represented in said Seq. ID Nos. 1 to 13 and 19, said nucleotide sequences of said nucleic acid coding said polypeptide and said hybridized nucleotide sequence, within the framework of degeneration of the genetic code.
wherein said method comprises the step of using said nucleic acid, a cell transfected with said nucleic acid, a cell transfected with a vector containing at least one copy of said nucleic acid, said polypeptide including the amino acid sequence represented in one of said Seq. ID Nos. 14 to 18 and 20 or said polypeptide coded by said nucleic acid.

18. A testing system for identifying effectors of a polypeptide, said polypeptide including an amino acid sequence, which is represented in one of Seq. ID Nos. 14 to 18 and 20 or which is coded by a nucleic acid consisting of a nucleic acid that codes said polypeptide, a nucleic acid that includes one of nucleotide sequences represented in Seq. ID Nos. 1 to 13 and 19, a nucleic acid having a hybridized nucleotide sequence hybridized with one of said nucleotide sequences represented in said Seq. ID Nos. 1 to 13 and 19 and said nucleotide sequences of said nucleic acid coding said polypeptide under stringent conditions, said hybridized nucleotide sequence coding for a polypeptide with biological activity of a co-modulator, or a nucleic acid having a degenerated nucleotide sequence corresponding to one of said nucleotide sequences represented in said Seq. ID Nos. 1 to 13 and 19, said nucleotide sequences of said nucleic acid coding said polypeptide and said hybridized nucleotide sequence, within the framework of degeneration of the genetic code; wherein
a) a reporter gene is expressed in a cell transfected with said nucleic acid or in a cell transfected with a vector containing at least one copy of said nucleic acid;
b) said cell, if said cell contains no nuclear receptor or only a small amount thereof, is also transfected with a vector containing DNA of the nuclear receptor;
c) said cell is cultured in the presence or absence of a plurality of test substances; and
d) a change in expression of said reporter gene is measured to determine which of said test substances is or are identified as an effector or effectors.

19. The testing system as defined in claim 18, wherein the nuclear receptor is an androgen receptor.

20. The testing system as defined in claim 18, wherein the cell is cultured in the presence or absence of the test substances and in the simultaneous presence of a ligand of the nuclear receptor.

21. The testing system as defined in claim 20, wherein the nuclear receptor is an androgen receptor and the ligand is an androgen.

22. A method for preparing a pharmaceutical agent, wherein
a) providing a testing system for identifying effectors of a polypeptide, said polypeptide including an amino acid sequence, which is represented in one of Seq. ID Nos. 14 to 18 and 20 or which is coded by a nucleic acid consisting of a nucleic acid that codes said polypeptide, a nucleic acid that includes one of nucleotide sequences represented in Seq. ID Nos. 1 to 13 and 19, a nucleic acid having a hybridized nucleotide sequence hybridized with at least one of said nucleotide sequences represented in said Seq. ID Nos. 1 to 13 and 19 and said nucleotide sequences of said nucleic acid coding said polypeptide under stringent conditions, said hybridized nucleotide sequence coding for a polypeptide with biological activity of a co-modulator, or a nucleic acid having a degenerate nucleotide sequence corresponding to one of said nucleotide sequences represented in said Seq. ID Nos. 1 to 13 and 19, said nucleotide sequences of said nucleic acid coding said polypeptide and said hybridized nucleotide sequence, within the framework of degeneration of the genetic code; wherein a reporter gene is expressed in a cell transfected with said nucleic acid or in a cell transfected with a vector containing at least one copy of said nucleic acid; said cell, if said cell contains no nuclear receptor or only a small amount thereof, is also transfected with a vector containing DNA of the nuclear receptor; said cell is cultured in the presence or absence of a plurality of test materials; and a change in expression of said reporter gene is measured to determine which of said test materials is or are identified as an effector or effectors;
b) substances are brought in contact with said testing system,
c) the action of the substances on the testing system is measured by comparison with controls,
d) one of the substances measured in step c) and showing modulation of activity of said polypeptide is identified, and
e) the one of the substances identified in step d) is mixed with formulation materials commonly used in pharmaceuticals to form said pharmaceutical agent.
